(19) United States
(12) Patent Application Publication
(10) Pub. No.: US 2009/0053221 A1
(43) Pub. Date: Feb. 26, 2009

(54) IMMUNE RESPONSE ENHANCING GLUCAN

(76) Inventors: Nai-Kong V. CHEUNG, Purchase, NY (US); Rolf Einar Engstad, Tromoso (NO)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/212,352

(22) Filed: Sep. 17, 2008

Related U.S. Application Data
(63) Continuation-in-part of application No. 12/161,285, filed on Jul. 17, 2008.
Application No. 12/161,285 is a continuation-in-part of application No. 11/334,763, filed on Jan. 17, 2006.

(51) Int. Cl. A61K 39/395; A61P 35/04

(52) U.S. Cl. 424/133.1; 424/130.1; 424/158.1; 424/172.1; 424/174.1

(57) ABSTRACT

This invention discloses a composition for enhancing the protective immunity in a subject, comprising an effective amount of a β-glucan and a vaccine, wherein the β-glucan enhances the immune response of the vaccine against cancer or infectious agents. The infectious agents can be viruses, fungi, bacteria or parasites. In one embodiment, the β-glucan is derived from yeast and comprises side chains attached to a β-(1,3) backbone. In another embodiment, the vaccine comprises an antibody and whole tumor cells. The invention also provides a method of enhancing protective immunity using said composition.

### CROSS-REFERENCE TO RELATED APPLICATIONS

This application is a Continuation-In-Part of U.S. Ser. No. 12/161,285, filed Jul. 17, 2008, which is the national stage application of International Application No. PCT/US07/01427, filed Jan. 17, 2007, which is a Continuation-In-Part of U.S. Ser. No. 11/334,763, filed Jan. 17, 2006. The contents of these prior applications are hereby incorporated in their entireties by reference in this application.

### STATEMENT REGARDING FEDERALLY SPONSORED RESEARCH OR DEVELOPMENT

This invention was funded in part by grants from the National Cancer Institute (CA61017, CA106450). Therefore, the government has certain rights in this invention.

Throughout this application, various references are cited. Disclosures of these publications in their entireties are hereby incorporated by reference into this application to more fully describe the state of the art to which this invention pertains.

### BACKGROUND OF THE INVENTION

Glucans are a heterogeneous group of glucose polymers found in the cell walls of plants, bacteria and fungi. The basic structure of branched β-1,3-glucan consists of a backbone of β-1,3-linked glucose molecules with β-1,6-linked side branches and/or β-1,3-linked side branches depending on the specific source of glucan.

β-glucans have been tested for tumor therapy in mice for nearly 40 years [1-2]. Several forms of mushroom-derived β-glucans are used clinically to treat cancer in Japan, including PSK (from Coriolus versicolor), Lentinan and Schizophyllan. In randomized trials in Japan, PSK has moderately improved survival rates in some cancer trials after gastrectomy [3-4], colorectal surgery [5-6], and esophagectomy [7] to remove primary tumors. Results have been less encouraging in breast cancer [8-9] and leukemia [10]. Schizophyllan also moderately improved survival of patients with operable gastric cancer [11], inoperable gastric cancer [12-13], and cervical cancer [14]. While β-glucans are not widely used by Western oncologists, β-glucan containing botanical medicines such as Reishi and maitake [15] are widely used by U.S. cancer patients as alternative/complementary cancer therapies.

In Europe and USA, β-glucans especially from Bakers' yeast have long been employed as feed additives for animals [16], as dietary supplement for humans [17], in treatment of wounds [18], and as an active ingredient in skin cream formulations. The basic structural unit in β-glucans of most of the organisms containing glucans are the β-1,3-linked glycosyl units. Glucans of different origin have usually a different composition of linkage types not necessarily being β-1,3-linked. This is the case for glucans derived from grains like barley where the glucan also includes β-1,4-linkages. Depending upon the source and method of isolation, β-glucans have also various degrees of branching and of linkages in the side chains, and some glucans do not even have a side chain but only one single glucose molecule attached to the main chain or they are simply linear glucans without any side chains or attached molecules at all. In short, glucans come in a large variety and shape. The frequency and hinge-structure of side chains is said to determine its immunomodulatory effect. β-glucans of fungal and yeast origin are normally insoluble in water, but can be made soluble either by acid hydrolysis or by derivatization introducing charged groups like phosphate, sulphate, amine, carboxymethyl and so forth to the molecule [19-20].

It is generally accepted that β-glucans of microbial origin, like yeasts, are recognized by specific pattern recognition receptors on immune cells as a result of phylogenetic adaptation for detecting possible pathogens. β-glucans in, e.g., fungal cell walls are major structural element that secure the strength and integrity of the cell and are thus vital for the organism. β-1,3-glucans are present in almost all fungal cells and they are highly conserved structures, the latter being a prerequisite for the so-called Pathogen Associated Molecular Patterns (PAMPs) recognized by the immune system. Immunologically active β-glucans are likely to bind to a β-glucan receptor like, for instance, Dectin-1 when introduced to the organism through the gastrointestinal tract.

Examples of useful β-glucans include, but are not limited to, particulate, semi-soluble and soluble yeast cell wall glucans as described in PCT/IB95/00265 and EP 0759089. Other β-1,3-glucan compositions having similar characteristics as described for yeast glucans, like specific preparations of, e.g., lentinan, scleroglucan and schizophyllan showing durable interchain interactions, are likely to be effective. β-glucans having β-1,3 side chains are also expected to be useful. Likewise, β-1,3-glucan formulations solublized by derivatization, like glucan phosphates, glucan sulphates, and carboxymethyl-glucans, which retain the immunopotentiating activity and interchain associations of the native molecule would be potential active products.

β-glucan formulations not presenting a pathogen-like feature could nevertheless be potent adjuvants for immunotherapy when administered systemically, like when given i.v. as described in Herlyn et al. (Monoclonal antibody-dependent murine macrophage-mediated cytotoxicity against human tumors is stimulated by lentinan. Jpn. J. Cancer Res. 76, 37-42 (1985)), or when given i.p. as described in U.S. Ser. No. 60/261,911.

Immunity is the state of being protected from a disease. It can be achieved by passive or active immunization. Passive immunization is the transfer of active humoral immunity in the form of antibodies or immune cells, from one individual to another. Passive immunization can occur naturally, as when maternal antibodies are transferred to the fetus through the placenta, or artificially, as when high levels of antibodies specific for a pathogen or toxin are transferred to an individual requiring immunity.

Active immunization entails the education of host's own immune cells to react against a molecule or target, typically carried on a foreign molecule and introduced into the body. In cellular immune response, cells of the immune system kill cells of the body that have been infected with a pathogen or that are cancerous. The first phase of the response, called the activation phase, involves activation and cell division of both helper T (T_{H}) and cytotoxic T (T_{C}) cells. The second phase of the response, called the effector phase, occurs when the activated T_{C} cells encounter and kill the target cells. Active immunization can occur naturally, as when a person comes in contact with, for example, a microbe, and then the person becomes immunized against the microbe. Artificial active immunization is where the microbe, or parts of it, is administered to the person. Vaccination is an active form of immunization.

The use of mAbs has become increasingly popular for the treatment of cancer. There are a number of mAbs approved by the FDA for use in solid tumors (e.g., breast, colon, lung cancer) and hematologic malignancies (e.g., leukemias, lymphomas). Antibodies may induce a complement mediated cytotoxicity or antibody-dependent cellular cytotoxicity towards tumors [21]. MAbs may also exert antitumor effects by inducing apoptosis [22], interfering with ligand-receptor interactions, or preventing the expression of proteins that are critical to the neoplastic phenotype [23].

Recent studies have provided strong evidence for the importance of the Fc domain in the efficacy of antitumor antibodies. In murine systems, Fc receptor (FcγR) engagement was required for efficacy of antitumor antibodies in several tumor antigen models, including HER-2 [24]. Several clinical studies have shown a positive correlation between the presence of favorable FcγR polymorphic alleles with higher affinities for IgG and improved clinical outcomes in mAb treated patients [25-27]. These studies have established that Fc-FcγR interactions are critical to antitumor antibody efficacy in the mouse and are correlative with clinical outcome in patients. In addition to their roles as opsonins, antitumor antibodies are predicted to enhance dendritic cell internalization and antigen presentation of tumor antigen via endocytosis and phagocytosis of tumor antigen-containing immune complexes and antibody-opsonized tumor target cells, respectively [28, 29].

β-Glucan as a biological response modifier has been known to modulate immune response through its effect on the natural immune system, mainly through interaction with myeloid cells (macrophages) and dendritic cells [35, 36]. Oral β-glucan enhances the direct anti-tumor effect of mAb in preclinical studies [37-39].

### SUMMARY OF THE INVENTION

This invention provides a composition for enhancing protective immunity in a subject, comprising an effective amount of a β-glucan and a vaccine, wherein said β-glucan has a β-(1,3) backbone and optionally β-(1,3) and/or β-(1,6) side chains, and wherein said β-glucan enhances the immune response induced by said vaccine against cancer or infectious agents.

In one embodiment of the invention, the vaccine is a cancer vaccine, and the immune response is against cancer. In another embodiment, the β-glucan has a numerical average molecular weight (NAMW) from about 6 kDa to about 30 kDa, wherein one or more β-glucan molecules form a higher order conformation, resulting in gelling and high viscosity profile.

In a further embodiment, the cancer vaccine comprises an antibody, and one or more components selected from the group consisting of whole tumor cells, tumor cell lysates, tumor cell derived RNAs, tumor cell derived proteins, tumor cell derived peptides, tumor cell derived carbohydrate, tumor cell derived lipids, tumor cell derived DNA sequences, and gene modified tumor cells. In yet another embodiment, the cancer vaccine comprises an antibody and whole tumor cells.

This invention also provides a method of enhancing protective immunity in a subject, comprising the steps of: (a) administering to the subject a vaccine; and (b) administering to the subject a β-glucan, wherein said β-glucan has a β-(1,3) backbone and optionally β-(1,3) and/or β-(1,6) side chains, and wherein said β-glucan enhances the immune response of the vaccine against cancer or infectious agents. The vaccine and β-glucan are administered at the same or different time.

In one embodiment of the invention, the vaccine is a cancer vaccine, and the immune response is against cancer. In another embodiment, the β-glucan has a numerical average molecular weight from about 6 kDa to about 30 kDa, wherein one or more β-glucan molecules form a higher order conformation, resulting in gelling and high viscosity profile.

In a further embodiment of the invention, the cancer vaccine comprises an antibody and one or more components selected from the group consisting of whole tumor cells, tumor cell lysates, tumor cell derived RNAs, tumor cell derived proteins, tumor cell derived peptides, tumor cell derived carbohydrate, tumor cell derived lipids, tumor cell derived DNA sequences, and gene modified tumor cells. In yet another embodiment, the cancer vaccine comprises an antibody and whole tumor cells.

Preferably, the β-glucan used in the above method is a yeast β-glucan having a numerical average molecular weight range from about 6,000 to about 30,000 Daltons, and calculated weighted average molecular weight (WAMW) in the range of 2×10⁵-3×10⁶ g/mol. The yeast β-glucan can be administered at the same or different time as the administration of the vaccine. Preferably, the yeast β-glucan is capable of priming or inducing secretion of cytokines, chemokines or growth factors.

### BRIEF DESCRIPTION OF THE FIGURES

FIG. 1. Structure of branched yeast β-1,3-glucans with β-1,3-linked side chains anchored to the main chain through β-1,6-linkages.

FIG. 2. ¹H NMR spectrum of a typical SBG™ (Soluble Beta Glucan) sample (Biotec Pharamacon ASA, Tromsø, Norway). A SBG™ sample was dissolved in DMSO-d₆ at a concentration of approximately 20 mg/ml and with a few drops of TFA-d added. The spectrum (cut-out from 2.7 to 5.5 ppm) was collected over 2 hours on a JEOL ECX 400 NMR spectrometer at 80° C. Chemical shifts were referenced to residual proton resonance from the DMSO-d₆ at 2.5 ppm, and the spectrum was baseline corrected.

FIG. 3. Viscosity profile of SBG™. Profiles for a 2% solution of SBG™ at 20 or 30° C. at different shear rates were shown. Glycerol (87%) was used as reference solution.

FIG. 4. 4A: Survival curves of groups of five mice treated with 3F8 mAb after iv challenge with syngeneic EL4 lymphoma cells. One single does of 200 μg of 3F8 mAb against GD2 administered at challenge or 1-10 days after 5×10⁴ tumor cells challenge. 4B: Survival curves of EL4 tumor survivors after 3F8 treatment re-challenged with iv EL4.

FIG. 5. Mouse serum anti-EL4 tumor antibody titers at week 8 after C57B/6 mice were immunized intravenously with 5×10⁴ irradiated or live EL4 lymphoma tumor cells with 200 μg tumor-reactive 3F8 mAb. Live tumor cells were mixed with 3F8 or given 2 hour before 3F8 by injection through the tail vein. Mouse serum anti-EL4 tumor antibody titers were assayed by ELISA using standard curve generated by 3F8. Data represent mean+standard error. Live cells with 3F8 generated a significant serum anti-tumor antibody response compared with control mice receiving 3F8 only (p<0.01) and a trend of higher serum antibody response was obtained with live cells than irradiated cells (p=0.344).

FIG. 6. Survival curves of C57B/6 mice re-challenged with 5×10⁴ EL4 cells iv after immunization intravenously with 5×10⁴ irradiated or live EL4 lymphoma tumor cells with 200 μg tumor-reactive 3F8 mAb. During vaccination, live tumor cells were mixed with Ab or given 2 hour before Ab by injection through the tail vein. Mice receiving live cells together with 3F8 survived significantly longer than control mice upon tumor iv re-challenge (p<0.05), comparable to irradiated cell or irradiated cells plus 3F8.

FIG. 7. Survival curves of C57B/6 mice re-challenged with 5×10⁴ EL4 cells iv after immunization subcutaneously with live or irradiated EL4 lymphoma tumor cells (5×10⁵) in the presence of tumor-reactive Ab 3F8 (50 μg) plus yeast β-glucan (YG, 2 mg). Mice received live EL4 and 3F8 survived longer than control (p<0.05) and mice received live EL4, 3F8 plus yeast β-glucan survived longer than either live EL4 plus 3F8 (p<0.001) or irradiated EL4 (p<0.05).

FIG. 8. Mouse serum anti-EL4 tumor antibody titers at week 4, 8 and 12 after C57B/6 mice were immunized subcutaneously with live EL4 lymphoma tumor cells (5×10⁵) in the presence of tumor-reactive Ab 3F8 (50 μg) plus yeast β-glucan (0.1-4 mg). Mouse serum anti-EL4 tumor antibody titers were assayed by ELISA using standard curve generated by 3F8. Data represent mean+standard error for 5 mice. Antibody titer against EL4 tumor cells correlates with the dose of yeast glucan.

FIG. 9. Balb/c mice were immunized subcutaneously with a mixture of RVE tumor cells (2×10⁶), tumor-reactive Ab 3F8 (50 μg) and yeast β-glucan (2 mg). Mouse serum antibody titers were assayed by FACS using standard curve generated by 3F8. Data represent mean+standard error for 5 mice. RVE/3F8/yeast glucan generates significantly higher antibody response than RVE alone (p<0.001).

FIG. 10. C57B/6 mice were immunized subcutaneously with EL4 lymphoma (5×10⁵) in the presence of tumor-reactive Ab 3F8 (50 μg) plus adjuvants: QS21 10 μg, GPI-0100 100 μg and yeast and barley glucan 2 mg. Mouse serum anti-tumor antibodies were assayed by FACS against EL4 using standard curve generated by 3F8. Data represent mean+standard error for 5 mice. The adjuvant effect of yeast glucan is comparable to QS21, but significantly better than no adjuvant control, GPI-0100 and barley glucan (p<0.001).

FIG. 11. Defines a range and specific values of the Degree of Polymerization (DP) and the average molecular weight (NAMW) of different batches of a preferred yeast β-glucan as used in the present invention.

FIG. 12. A typical chromatogram showing the calculated weighted average molecular weight (WAMW) of the Biotec Pharmacon ASA glucan SBG™ in the range of 2×10⁵-3×10⁶ g/mol.

### DETAILED DESCRIPTION OF THE INVENTION

The following terms shall be used to describe the present invention. In the absence of a specific definition set forth herein, the terms used to describe the present invention shall be given their common meaning as understood by those of ordinary skill in the art.

In the present invention, the expression “higher order conformation” refers to the three-dimensional shape formed by two or more glucan molecules interacting with one another and establishing relatively stable interchain associations through hydrogen bonds.

Adjuvants as used herein are pharmacological or immunological agents that modify the effect of other agents, such as drugs or vaccines.

The term “animal” is used to describe an animal, preferably a mammal, more preferably a human, to whom treatment or method according to the present invention is provided.

As used herein, the term “pharmaceutically acceptable carrier, additive or excipient” means a relatively safe substance which, when combined with a therapeutic composition, may facilitate the administration of the composition to animals, preferable mammals, and most preferably humans.

In the present invention, the term “immunostimulating” refers to stimulation of the immune system by inducing activation or increasing activity of any components of the immune system.

In the present invention, the term “immunopotentiating” refers to the ability of a substance to enhance or increase the immunostimulating effect of another substance.

The term “cancer” refers to pathological process that results in the formation and growth of a cancerous or malignant neoplasm, and includes, but is not limited to, neuroblastoma, melanoma, non-Hodgkin's lymphoma, Epstein-Barr related lymphoma, Hodgkin's lymphoma, retinoblastoma, small cell lung cancer, brain tumors, leukemia, epidermoid carcinoma, prostate cancer, renal cell carcinoma, transitional cell carcinoma, breast cancer, ovarian cancer, lung cancer colon cancer, liver cancer, stomach cancer, and other gastrointestinal cancers.

The term “effective amount” is used to describe that amount of a compound, when administered to an animal or a human, would lead to a desirable effect, such as suppression or eradication of tumor growth or spread of a cancer, or some desirable immune responses. When the administration of two requisite components is necessary to achieve a desirable biological effect, the effective amount of each component may be different, and refers to the amount that, after the two components are administered, will produce the expected effect.

Glucans as used herein are glucose polymers found in the cell walls of plants, bacteria and fungi. A β-1,3-glucan may be linear or branched. A linear β-1,3-glucan consists of a backbone of β-1,3-linked glucoses, while a branched β-1,3-glucan has basically a backbone of β-1,3-linked glucoses and side chains linked to the backbone via β-1,6 linkages, wherein the glucoses in the side chains may be β-1,3-linked and/or β-1,6-linked. In one embodiment of this invention, the side chain glucoses of a branched β-1,3-glucan are predominantly β-1,3-linked.

The Numerical average molecular weight (NAMW) range of the β-glucans is determined by using the method of Nelson & Somogyi (Nelson, N., 1944, “A Photometric Adaptation of the Somogyi Method for the Determination of Glucose”, J. Biol. Chem., 153:375-380; Somogyi, M., 1937, “A Reagent for the Copper-Iodometric Determination of Very Small Amounts of Sugar”, J. Biol. Chem., 117:771-776; Somogyi, M., 1952, “Notes on Sugar Determination”, J. Biol. Chem., 195:19-23). This is a method that determines reducing sugar via a reaction with a copper reagent and subsequent photometric detection, and it is used to quantify the concentration of reducing ends in the samples. The average degree of polymerization (DP) is obtained by dividing the total carbohydrate concentration by the concentration of reducing ends, and the average molecular weight can then be determined by the formula NAMW=(DP×162)+18. The degree of polymerization (DP) in a polymer molecule is the number, n, of repeating units in the polymer chain. The Numerical Average Molecular Weight (NAMW) as used in this specification is the total weight of all the polymer molecules in a sample, divided by the total number of polymer molecules in a sample.

Molecular weight measurements that depend on the contributions of molecules according to their sizes give Weighted Average Molecular Weights (WAMW). Light scattering and ultracentrifuge methods are examples of this type of technique. The Weighted Average Molecular Weight is larger than or equal to the Numerical Average Molecular Weight. The two parameters can be represented by the formulas:

$$W A M W = {M}_{W} = \frac{{\sum }_{i} {N}_{i} {M}_{i}^{2}}{{\sum }_{i} {N}_{i} {M}_{i}}$$

$$N A M W = {M}_{n} = \frac{{\sum }_{i} {M}_{i} {N}_{i}}{{\sum }_{i} {N}_{i}}$$

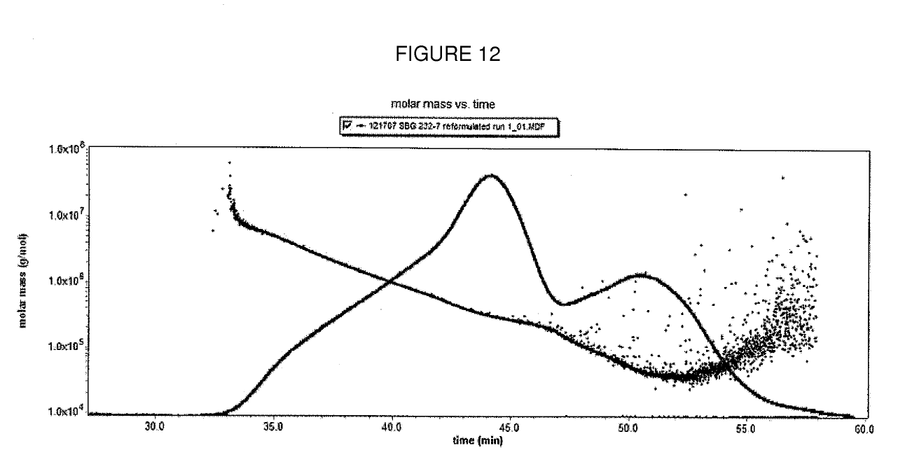

The Weighted Average Molecular Weight of the β-glucans is determined by GPC-MALLS analyses. The GPC-MALLS analyses are performed on the samples in aqueous solution, and the obtained MW reflects the weight of the macromolecular structures found in solution, which do not necessarily consist of only single chains. A typical chromatogram of the Biotec Pharmacon ASA glucan SBG™ with MW data is shown in FIG. 12.

Many β-glucans may be used in this invention, particularly those having β-1,3 side chains, and β-1,3-glucans isolated or derived from yeast. In one embodiment of the present invention, the glucan has a numerical average molecular weight range of from about 6 to 30 kDa, while the calculated weighted average molecular weight (WAMW) range of the Biotec Pharmacon ASAs glucan product SBG™ using GPC-MALLS analyses is in the range from 2×10⁵ g/mol to 3×10⁶ g/mol. In another embodiment, the β-glucan shows interchain associations, giving rise to a higher order conformation as manifested by gelling and a high viscosity profile.

The ability of β-glucans to have immunopotentiating activity is likely the result of their ability to present multiple epitopes for interaction with receptors on the target cells, thereby clustering β-glucan receptors and mimicking the challenge by a pathogenic organism. Such multiple interactions with specific receptors on the cell are believed to depend partly on the glucans' ability to form “higher order” conformation presenting multiple binding epitopes in close vicinity. Soluble β-glucan formulations which possess durable interchain associations, as manifested by a high viscosity profile, would thus be likely candidates for possessing “limmunpotentiating” abilities.

The term “vaccine” as used herein is a preparation used to enhance protective immunity against cancer, or infectious agents such as viruses, fungi, bacteria and parasites. Such a vaccine is useful as a prophylactic agent, although it can also be used to treat a disease. Vaccines contain cells or antigens which, when administered to the body, cause an immune response with the production of antibodies and immune lymphocytes (T-cells). Vaccines have been widely used to control and even eradicate infectious diseases such as polio and smallpox.

The term “cancer vaccine” refers to a vaccine that induces an immune response against a particular cancer. Cancer vaccines can be categorized as: antigen vaccines, whole cell vaccines, dendritic cell vaccines, DNA vaccines and anti-idiotype vaccines. To date, there are a few FDA licensed cancer prevention vaccines. These include (1) vaccine to protect against infection with the human papilloma virus (HPV) to prevent cervical cancer, (2) hepatitis B vaccine to protect against infection with the human Hepatitis B virus to prevent hepatocellular carcinoma, and (3) melanoma vaccine for canines.

Examples of cancer vaccines as used herein include whole tumor cells, tumor cell lysates, tumor cell derived RNAs, tumor cell derived proteins, tumor cell derived peptides, tumor cell derived carbohydrates, tumor cell derived lipids, and tumor cell derived DNA sequences. These tumor cells could be derived from a patient's own tumor or tumor from an unrelated donor. One potential advantage of cell-based vaccines is that they contain a wide range of antigens. A cancer vaccine may prevent further growth of existing cancer, protect against recurrence of treated cancer, or eliminate cancer cells not already removed by other treatments.

“Whole cell tumor vaccines”, also referred to as “whole tumor vaccines” comprise tumor cells which may be autologous or allogeneic for the patient. These cells comprise cancer antigens which can stimulate the body's immune system. As compared to the administration of individual cancer antigens, a whole cell exposes a large number of cancer specific (unique or up-regulated) antigens to the patient's immune system. This stimulation of the immune system means that the patient is better able to prevent the subsequent growth or establishment of a tumor.

Whole cell tumor vaccines, which have been used to treat pancreatic and prostate cancers, typically comprise tumor cells which have been modified in vitro, e.g., irradiated and dead tumor cells are preferred in many applications, although live tumor cells may be used in the vaccine. The whole cell vaccine may comprise intact cells but a cell lysate may alternatively be used, and “whole” cell should be understood with this in mind. The use of such a lysate (or intact cell preparation) means that the vaccine will comprise in excess of 10 antigens, typically in excess of 30 antigens.

Active immunity as used herein is a type of immunity or resistance developed in a host as a result of its own production of antibodies or cellular immune response following an exposure to an antigen or vaccine. Active immunity is usually long-lasting.

Infection as used herein refers to an invasion by pathogenic micro-organisms of a bodily part in which conditions are favorable for growth, production of toxins, and resulting injury to tissue.

Protective immunity is generated when the natural ability of the body's immune system to resist growth or establishment of a tumor is enhanced. Such protection may be achieved against a tumor type which has not yet developed in the subject. Thus, a patient with a family history of a certain cancer, e.g. prostate cancer, may be protected against development of that cancer before any cancerous cells or abnormalities indicative of cancer have been observed—the classic vaccination model. Alternatively or in addition, protection may be desired against tumors derived, e.g., by metastasis, from a known primary tumor. Such secondary tumors may be present in the body at the time the vaccine is administered. Another scenario would be protective immunity against subsequent development of a further primary tumor in a patient who has already been diagnosed with, and typically received treatment for, a primary tumor. In the present invention, it is shown that therapeutic antibodies not only provide passive immunotherapy through antibody-dependent tumor cell cytotoxicity but also can promote active immunity. Similarly, protective immunity can be generated in a subject against infection by an infectious agent before or after the agent has entered the subject.

In one embodiment of the present invention, the cancer vaccine may include a second component such as an antibody. The antibody may be a monoclonal antibody, or an antibody against cancer or tumor cells, which include but are not limited to anti-CEA antibody, anti-CD20 antibodies, anti-CD25 antibodies, anti-CD22 antibodies, anti-HER2 antibodies, anti-tenascin antibodies, MoAb M195, Dacluzimab, anti-TAG-72 antibodies, R24, Herceptin, Rituximab, 528, IgG, IgM, IgA, C225, Epratuzumab, MoAb 3F8, and antibody directed at the epidermal growth factor receptor, or a ganglioside, such as GD3 or GD2. In another embodiment, the antibody is a tumor-binding antibody. The antibody should be able to bind to Fc receptors. Preferably, the antibody is capable of activating complement and/or activating antibody dependent cell-mediated cytotoxicity. In a further embodiment, the antibody modulates the cellular immune response.

Antibodies as used herein refer to any part of immunoglobulin molecules (e.g. a monoclonal antibody) having specific cancer cell binding affinity by which they are able to exercise antitumor activity. Examples are antigen binding fragments or derivatives of antibodies. Furthermore, the antibody used in the present invention can be a single monoclonal antibody or a combination of antibodies. The antibodies may be directed to at least one epitope or multiple epitopes of an antigen or multiple antigens. Accordingly, this invention encompasses at least one antibody. An opsonising antibody is one

The cancer recognized by antibodies includes, but is not limited to, neuroblastoma, melanoma, non-Hodgkin's lymphoma, Epstein-Barr related lymphoma, Hodgkin's lymphoma, retinoblastoma, small cell lung cancer, brain tumors, leukemia, epidermoid carcinoma, prostate cancer, renal cell carcinoma, transitional cell carcinoma, breast cancer, ovarian cancer, lung cancer colon cancer, liver cancer, stomach cancer, and other gastrointestinal cancers.

It will be recognized by one of ordinary skills in the art that the various embodiments of the invention relating to specific methods of treating tumors and cancer disease states may relate within context to the treatment of a wide number of other tumors and/or cancers not specifically mentioned herein. Thus, it should not be construed that embodiments described herein for the specific cancers mentioned do not apply to other cancers.

The present invention provides a composition for enhancing protective immunity in a subject, comprising an effective amount of a yeast β-1,3-glucan and a vaccine, wherein the β-1,3-glucan enhances the immune response induced by the vaccine and initiates protective immunity in such a subject. The immunity can be against cancer or infections. In one embodiment, the β-1,3-glucan contains side chains of β-1,3-linked glucose units attached to the backbone via β-1,6-glycosidic bonds. In another embodiment, the β-1,3-glucan is a mixture of linear and branched β-1,3-glucans. In a further embodiment, the vaccine is a cancer vaccine comprising whole tumor cells and an antibody.

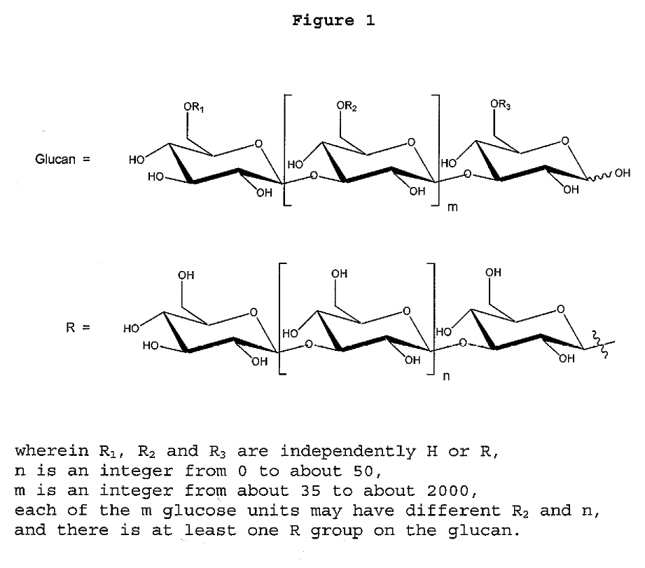

An example of a highly active composition of yeast β-1,3-glucans is a mixture of soluble β-1,3-glucan chains with numerical average molecular weight (NAMW) >6000 Daltons that interact to give a higher order conformation. In one embodiments the mixture of soluble β-1,3-glucans have an NAMW >6000 Da, preferably, an NAMW ranging from 6000-30,000 Da, with β-1,3 linked side chain(s) extending from the main chain via β-1,6 linkages as shown in FIG. 1.

In one embodiment of the present invention, the β-glucan composition comprises yeast β-1,3-glucans derived from yeast cell walls which have been treated by a hydrolyzing agent like for instance acid or enzyme to significantly reduce or eliminate (1,6) linkages within the glucan branches (a single (1,6) link is required to form the branch). Thus, preferably less than 10%, more preferably less than 5%, most preferably less than 3% or 2% of the glycosidic bonds in the molecule will be (1,6) linkages. These products can be particulate, semi-soluble, soluble or a gel.

An example of a soluble hydrolyzed product for use in the present invention are soluble yeast product like the pharmaceutical-grade product SBG™ (Soluble Beta Glucan) as produced by Biotec Pharmacon ASA, a Norway based company.

The product is an underivatized (in terms of chemical modifying groups) aqueous soluble β-1,3/1,6-glucan, characterised by NMR and chemical analysis to consist of polymers of β-1,3-linked D-glucose containing side-chains of β-1,3 and β-1,6-linked D-glucose, wherein the number of β-1,6 moieties in the side chains (not including at the backbone/side chain branch point) is considerably reduced as compared to the structure of said glucan in the yeast cell wall. An example of such a composition is as follows:

| COMPOSITION | Value/range | | typical value |
|---|---|---|---|
| WATER | 977-983 | gram/kg | 980 |
| CARBOHYDRATES | 18-22 | gram/kg | 20 |
| PROTEINS | max 1 | gram/kg | <1 |
| ASH | max 1 | gram/kg | <1 |
| LIPID | Max 1 | gram/kg | <1 |

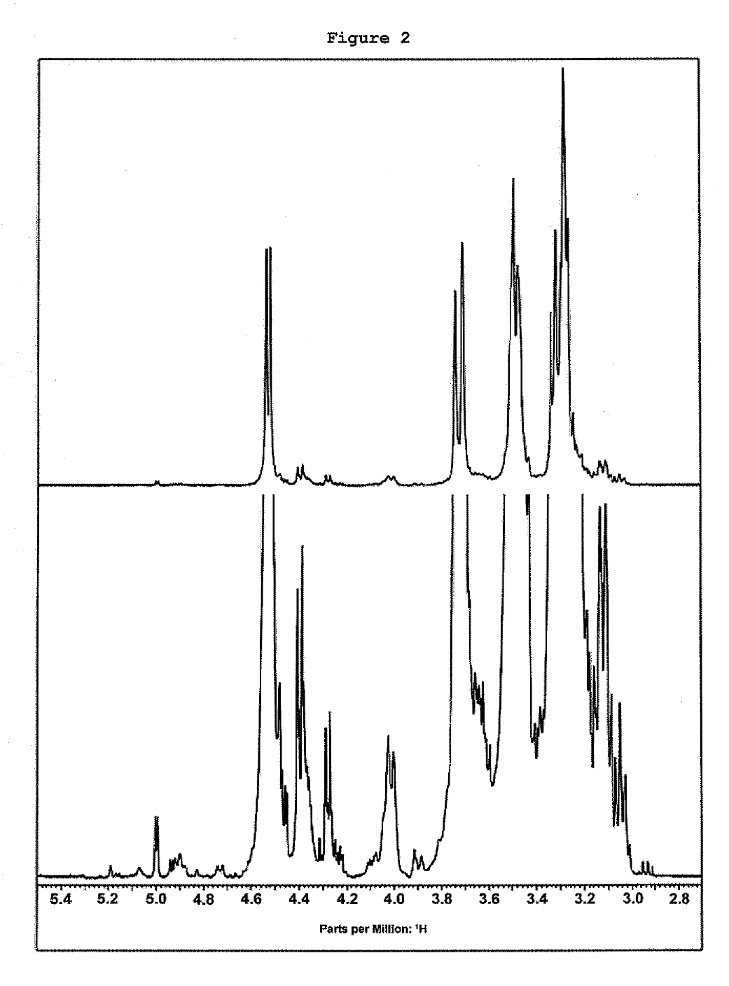

The molecular structure of SBG™ is as shown in FIG. 2.

SBG™ (Soluble Beta Glucan) as produced by Biotec Pharamacon ASA (Tromsø, Norway) is an un-derivatized aqueous soluble β-1,3-1,6-glucan characterized by NMR and chemical analysis to consist of a linear β-1,3-glucan backbone having side chains of β-1,3-linked D-glucose units wherein the side chains are attached to the backbone via β-1,6-linkages (see FIG. 1).

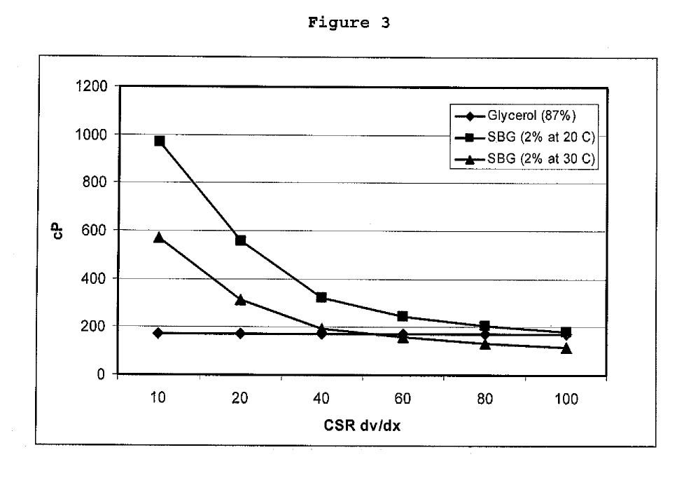

As shown in FIG. 1, SBG™ shows a complex β-glucan composition with high molecular weight chains having β-1,3-linked side chains attached to the repeating β-1,3-linked main chain through a β-1,6-linked branching point. SBG™ presents durable interchain associations as demonstrated by its high viscosity profile and gelling behavior (see FIG. 3).

A preferred glucan containing formulation for use in the invention is a mixture of soluble β-glucan molecules with numerical average molecular weights (NAMW) >6000 Daltons that interact to give a higher order conformation. For example, a mixture of linear β-1,3-glucan chains with an NAMW of >6 kDa, preferably with an NAMW ranging from 6-30 kDa, with β-1,3 linked side chain(s) extending from within the main chain as shown in FIG. 1.

Most preferably, the β-glucans have an average molecular weight of about 15-20 kDa, with a range from about 6 to about 30 kDa, preferably from about 10 to about 25 kDa.

The most preferred β-glucans used in accordance with the present invention have utility as safe, effective, therapeutic and/or prophylactic agents, either alone or as adjuvants, to enhance the immune response in humans and animals by amongst other effects inducing a local inflammatory response by stimulating or priming the systemic immune system to release certain biochemical mediators (e.g., IL-1, IL-3, IL-6, IL-17, TNF-α, and GM-CSF). This specific effect is unique to these β-glucans while similar glucans claim not to stimulate or prime the immune system in that manner. SBG™ has been shown to be a potent immunostimulating agent for activating human leukocytes in vitro, e.g., priming and inducing the production of cytokines (see Engstad et al., 2002, “The effect of soluble β-1,3-glucan and lipopolysaccharide on cytokine production and coagulation activation in whole blood”, Int. Immunopharmacol. 2:1585-1597), and also for modulating immune functions when given p.o. (see Breivik et al., 2005, “Soluble β-1,3-1,6-glucan from yeast inhibits experimental periodontal disease in Wistar rats”, J. Clinical Periodontology, 32:347-353). It is preferable for the yeast glucans of the present invention to have such functional properties of priming and inducing cytokine production by human leukocytes.

Suitable forms of yeast glucans include, but are not limited to, particulate, semi-soluble, soluble or gel form.

In one embodiment, a product for use in connection with the present invention is NBG™ (Norwegian Beta Glucan), a particulate yeast product as produced by Biotec Pharmacon ASA. NBG® is a product derived from Bakers Yeast (Saccharomyces cerevisiae). The product is a natural underivatized (in terms of chemical modifying groups) particulate β-1,3/1,6-glucan, characterised by NMR and chemical analysis to consist of polymers of β-1,3-linked D-glucose containing side-chains of β-1,3 and β-1,6-linked D-glucose. NBG® is a purified, yeast cell wall preparation which is produced by removing the mannan protein outer layer thus concentrating the glucan content basically not retaining the glucan's in vivo morphology. Generally, NBG® has particles of 1 micron or greater. Furthermore, NBG® and similar compositions actively prime, stimulate and/or induce immune system mediators like pro-inflammatory cytokines, such as IL-1 and TNF.

Typical values for the chemical composition of NBG® are as follows:

| COMPOSITION | % by weight | Typical range |
|---|---|---|
| CARBOHYDRATES | Min 75 | 75-80 |
| LIPIDS | Max 5 | 3-5 |
| NITROGEN | Max 1.4 | 0.8-1.2 |
| ASH | Max 12 | 8-10 |
| TOTAL SOLID | Min 95 | 95-98 |

Another example of glucans is WGP 3-6 which is a product of Whole Glucan Particles containing β-(1,3)-(1,6)-glucan and is a purified, yeast cell wall preparation. Whole Glucan Particles are produced by removing the mannan protein outer layer and exposing the β-glucan while retaining the glucan's in vivo morphology. The Whole Glucan Particles may have particle size of 1 micron or greater. Such Whole Glucan Particles may be obtained from any glucan-containing fungal cell wall source, but the preferred source is Saccharomyces cerevisiae. Whole Glucan Particles usually do not induce pro-inflammatory cytokines, but such an effect can not be excluded at this point.

Other structures and/or structural conformations in the composition of β-1,3-glucans as described above can be readily identified or isolated by a person of ordinary skill in the art following the teaching of this invention, and is expected to have similar therapeutic effect when administered through different routes other than orally. The above is thus a guideline to achieve a highly potent product, but is not a limitation towards even more potent products. Isolated structural elements of the complex mixture as described above are expected to have improved effects over the present formulation when administered orally.

Products having the desired structural features and showing a higher order conformation like SBG™ that facilitates the needed interaction with responding cells in the intestinal tract would be the preferred products when administered orally. Their action as immunopotentiators in synergy with anti-cancer antibodies is likely to be at least as powerful when administered parenterally, e.g., when administered intraperitoneally, subcutaneously, intra-muscularly or intravenously. Functional dose range of the glucans can be readily determined by one of ordinary skills in the art. For example, when administered orally the functional dose range would be in the area of 1-500 mg/kg/day, more preferable 10-200 mg/kg/day, or most preferable 20-80 mg/kg/day. When administered parenterally, the functional dose range would be 0.1-10 mg/kg/day.

In this invention, an appropriate β-1,3-glucan is used in combination with a tumor antigen presenting entity. In one embodiment, the tumor antigen presenting entity is a cancer vaccine, which may comprise whole tumor cells and an antibody. In one embodiment, the β-1,3-glucan is administered in the amount of 0.1-4 mg. In another embodiment, the antibody is administered in the amount of 10-1000 μg, and preferably 50 μg. In a further embodiment, the whole tumor cells are administered in the amount of 10⁵-10⁷ cells, and preferably 5×10⁵ cells.

The invention also provides a method of treating a subject with cancer, comprising the steps of: (a) administering to the subject a cancer vaccine; and (b) administering to the subject a yeast β-glucan, wherein the glucan exhibits adjuvant activity to the cancer vaccine. In one embodiment, the β-glucan and cancer vaccine are administered concurrently or sequentially, orally, subcutaneously or intravenously. In another embodiment, both the β-glucan and cancer vaccine are administered together subcutaneously.

Glucans derived from cell walls of yeasts, such as Saccharomyces cerevisiae, may be used in the above-described compositions. Preferably, glucans having β-1,3 and β-1,6 linkages, such as SBG™ (Soluble Beta Glucan) produced by Biotec Pharamacon ASA (Tromsø, Norway), is used in the above-described compositions. The above mentioned pharmaceutical compositions may contain pharmaceutically acceptable carriers and other ingredients known to enhance and facilitate drug administration. The relative amounts of the active ingredient, the pharmaceutically acceptable carrier, and any additional ingredients in a pharmaceutical composition of the invention will vary, depending upon the identity, size, and condition of the subject treated.

Such a pharmaceutical composition may comprise the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in forms which are generally well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. Controlled- or sustained-release formulations of a pharmaceutical composition of the present invention may be made using conventional technology.

The present invention also provides a composition comprising an effective amount of β-1,3-1,6-glucan capable of enhancing the efficacy of vaccines. In one embodiment, the vaccine is against cancer or infectious agents, such as bacteria, viruses, fungi, or parasites.

The present invention also provides a composition comprising an effective amount of β-1,3-1,6-glucan capable of enhancing host immunity. The host immunity includes, but is not limited to, antitumor immune responses.

This invention also provides kits for inhibiting cancer cell growth and/or metastasis. The invention includes a kit or an administration device comprising a glucan as described herein and information material which describes administering the glucan or a composition comprising the glucan to a human. The kit or administration device may have a compartment containing the glucan or the composition of the present invention. As used herein, the “Information material” includes, but is not limited to, a publication, a recording, a diagram, or any other medium of expression which can be used to communicate the usefulness of the composition of the invention for its designated use.

Typically, dosages of the compound of the present invention administered to an animal, preferably a human, will vary depending upon any number of factors, including but not limited to, the type of animal and type of cancer and disease state being treated, the age of the animal, the route of administration and the relative therapeutic index.

The route(s) of administration will be readily apparent to the skilled artisan and will depend upon any number of factors including the type and severity of the disease being treated, the type and age of the human patient being treated, and the like.

Formulations suitable for oral administration of the β-glucan include, but are not limited to, an aqueous or oily suspension, an aqueous or oily solution, an emulsion or as a particulate formulation. Such formulations can be administered by any means including, but not limited to, soft gelatin capsules.

Liquid formulations of a pharmaceutical composition of the present invention which are suitable for oral administration may be prepared, packaged, and sold either in liquid form or in the form of a dry product intended for reconstitution with water or other suitable vehicle prior to use. Administration can be by a variety of different routes including intravenous, subcutaneous, intranasal, buccal, transdermal and intrapulmonary. One of ordinary skills in the art would be able to determine the desirable routes of administration, and the kinds of formulations suitable for a particular route of administration.

In general, the β-glucan can be administered to an animal as frequently as several times daily, or it may be administered less frequently, such as once a day. The antibody treatment will for instance depend upon the type of antibody, the type of cancer, the severity of the cancer, and the condition of each patient. The β-glucan treatment is closely interrelated with the antibody treatment regimen, and could be ahead of, concurrent with, or after the antibody administration. The frequency of the β-glucan and antibody dose will be readily apparent to the skilled artisan and will depend upon any number of factors, such as, but not limited to, the extent and severity of the disease being treated, and the type and age of the patients. In one embodiment of the invention, the β-glucan is administered subcutaneously at or around the same time as the vaccine injection, in order to prime the antigen-presenting cells.

When administered orally, glucan is taken up by macrophages and monocytes that carry these carbohydrates to the marrow and reticuloendothelial system from where they are released, in an appropriately processed form, onto myeloid cells including neutrophils and onto lymphoid cells including natural killer (NK) cells. The processed glucan binds to CR3 on these neutrophils and NK cells, and activating their antitumor cytotoxicity in the presence of tumor-specific antibodies. The invention will be better understood by reference to the Experimental Details which follow, but those skilled in the art will readily appreciate that the specific experiments detailed are only illustrative, and are not meant to limit the invention as described herein, which is defined by the claims which follow thereafter.

The present invention provides a composition for enhancing protective immunity against cancer in a subject, comprising:
(a) a vaccine comprising an antibody and one or more components selected from the group consisting of whole tumor cells, tumor cell lysates, tumor cell derived RNAs, tumor cell derived proteins, tumor cell derived peptides, tumor cell derived carbohydrate, tumor cell derived lipids, tumor cell derived DNA sequences, and gene modified tumor cells; and
(b) a β-glucan having β-(1,3) side chains.
   In one embodiment of the composition, the β-glucan is derived from yeast. In another embodiment, the side chains of said β-glucan are attached to a β-(1,3) backbone via β-(1,6) linkages. In a further embodiment, the β-glucan has a numerical average molecular weight from about 6 kDa to about 30 kDa, and a weighted average molecular weight (WAMW) of 2×10⁵-3×10⁶ g/mol, and wherein one or more β-glucan molecules form a higher order conformation, resulting in gelling and high viscosity profile. In yet another embodiment, the β-glucan is capable of priming or inducing secretion of cytokines, chemokines or growth factors. In one embodiment of the composition, the antibody binds to the Fc receptor or activates complement. In another embodiment, the antibody is selected from the group consisting of anti-CEA antibody, anti-CD20 antibody, anti-tenascin antibody, anti-TAG-72 antibody, M195 antibody, DACLUZIMAB, R24 antibody, HERCEPTIN, RITUXIMAB, 528 antibody, IgG antibody, IgM antibody, IgA antibody, C225 antibody, EPRATUZUMAB, 3F8 antibody, an antibody directed at the epidermal growth factor receptor, anti-ganglioside antibody, anti-GD3 antibody, and anti-GD2 antibody. In still another embodiment, the antibody binds to cancer cells expressing an antigen selected from the group consisting of CD20, HER2, EGFR, GD2, and GD3.

The present invention also provides a method of enhancing protective immunity against cancer in a subject, comprising the steps of:
(a) administering to the subject a vaccine comprising an antibody; and
(b) administering to the subject a β-glucan having β-(1,3) side chains, wherein cancer growth in said subject is treated or prevented.
   This method may also be used to protect against biologic toxins, allergens, pathologic proteins (e.g. prions), and pathologic RNA or DNA. In one embodiment of the method, the antibody is an opsonising antibody. In another embodiment the vaccine further comprises one or more components selected from the group consisting of whole tumor cells, tumor cell lysates, tumor cell derived RNAs, tumor cell derived proteins, tumor cell derived peptides, tumor cell derived carbohydrate, tumor cell derived lipids, tumor cell derived DNA sequences, and gene modified tumor cells. In a further embodiment, the β-glucan is derived from yeast. In one embodiment, the side chains of said β-glucan are attached to a β-(1,3) backbone via β-(1,6) linkages. In another embodiment, said β-glucan has a numerical average molecular weight from about 6 kDa to about 30 kDa, and a weighted average molecular weight (WAMW) of 2×10⁵-3×10⁶ g/mol, and wherein one or more β-glucan molecules form a higher order conformation, resulting in gelling and high viscosity profile. In still another embodiment, said β-glucan is capable of priming or inducing secretion of cytokines, chemokines or growth factors. The cancer is neuroblastoma, melanoma, non-Hodgkin's lymphoma, Epstein-Barr related lymphoma, Hodgkin's lymphoma, retinoblastoma, small cell lung cancer, brain tumors, leukemia, epidermoid carcinoma, prostate cancer, renal cell carcinoma, transitional cell carcinoma, breast cancer, ovarian cancer, lung cancer colon cancer, liver cancer, stomach cancer, and other gastrointestinal cancers. In one embodiment of the method, the antibody binds to the Fc receptor or activates complement. In another embodiment, the antibody is selected from the group consisting of anti-CEA antibody, anti-CD20 antibody, anti-tenascin antibody, anti-TAG-72 antibody, M195 antibody, DACLUZIMAB, R24 antibody, HERCEPTIN, RITUXIMAB, 528 antibody, IgG antibody, IgM antibody, IgA antibody, C225 antibody, EPRATUZUMAB, 3F8 antibody, an antibody directed at the epidermal growth factor receptor, anti-ganglioside antibody, anti-GD3 antibody, and anti-GD2 antibody. In yet another embodiment, the antibody binds to cancer cells expressing an antigen selected from the group consisting of CD20, HER2, EGFR, GD2, and GD3. The vaccine and glucan are administered orally, intravenously, subcutaneously, intramuscularly, intraperitoneally, intra-nasally or transdermally, concurrently or sequentially.

### EXAMPLE 1

### Yeast β-Glucan Enhances Immune Responses

Whole tumor vaccines can induce tumor-specific protective immunity in preclinical tumor models. Recent clinical trials using GM-CSF-modified allogeneic or syngeneic tumor lines have yielded positive although modest clinical responses. When one reviews successful vaccines in human medicine, evidence continues to point to the importance of antibodies in both the induction as well as the maintenance of protective immunity. The persistence of cancer remission long after the completion of monoclonal antibodies strongly suggests an active immunity induced by “passive antibody therapy”. It is postulated that tumor vaccines when opsonized with specific antibodies will enhance their presentation to antigen presenting cells. In the presence of β-glucan, the efficacy of such vaccines can be further improved.

The EL4 syngeneic mouse model of lymphoma was used to study antibody response to whole tumor vaccine in the presence of β-glucan. When live EL4 tumor cells were planted subcutaneously or intravenously in immunocompetent C57Bl/6 mice, they engrafted rapidly causing death from large tumor masses and metastases to distant organs. When EL4 tumor cells were planted subcutaneously or intravenously in the presence of anti-GD2 antibody 3F8, tumor cell engraftment diminished. When challenged later with EL4 cells, there was marginal protective immunity. Since β-glucan is known to activate antigen-presenting cells, EL4 cells were administer in the presence of 3F8 as a tumor vaccine to test if β-glucan can provide adjuvant effect to induce protective immunity.

C57Bl/6 mice were vaccinated subcutaneously with EL4 lymphoma (as whole tumor vaccine) in the presence of anti-GD2 antibody 3F8 plus yeast β-glucan. Mouse sera were obtained at week 2, 4, and 8 after vaccination. Serum antibodies against surface antigens on EL4 cells were assayed by flow cytometry. Antibodies against total cell antigens (surface and cytoplasmic) were assayed by ELISA using EL4 cells bound to microtiter plates.

Results from these experiments indicate that: (1) 3F8 was necessary to prevent subcutaneous EL4 tumor engraftment; (2) 3F8 enhanced antibody response to EL4 whole tumor vaccine; (3) live EL4 tumor vaccine stimulated a significantly higher immune response compared to irradiated EL4 tumor vaccine; (4) antibody titer against EL4 tumor increased with increasing dose of glucan as an adjuvant, with an optimal dose at 2 mg; and (5) the higher the dose of glucan, the longer the mice were protected when subsequently challenged with intravenous EL4 in a tumor prevention model.

### EXAMPLE 2

### Yeast β-Glucan Enhances Immune Responses

The combination of tumor cell and anti-tumor mAb may be potentially useful as a whole cell tumor vaccine. The model vaccine used in the current study is the EL4 tumor and 3F8 antibody combination. 3F8 is a murine IgG3 anti-GD2 mAb; in patients with metastatic neuroblastoma, 3F8 was previously shown to prolong survival [27, 30, 31]. IgG3 antibody in general has also been shown to enhance immunity and memory response and the effect is highly dependent on its ability to activate complement. A possible mechanism is the increase of B-cell activation caused by immune complexes co-crosslinking the B-cell receptor with the complement receptor 2 (CR2)/CD19 receptor complex, which is known to lower the threshold for B-cell activation [32]. The mouse lymphoma EL4 expresses high level of GD2 ganglioside and can be treated effectively with 3F8 mAb [33]. The protection against EL4 by 3F8 antibody therapy was unaffected in mice deficient in C3 or complement receptor 3 (CR3) but was almost completely abrogated in FcγRI/III-deficient mice [34].

### Materials and Methods

mAbs and Reagents

The mAb 3F8 (IgG3) against GD2 was previously described [40]. Yeast and barley β-glucans were provided by Biotec Pharmacon (Tromsø, Norway) and Megazyme (Bray, Ireland), respectively. HB11 anti-H2b IgG2a mAb (ATCC, Manassas, Va.) was used as a control antibody. The L3T4 (GK1.5) anti-CD4 mAb (ATCC) was used to deplete mouse CD4⁺ T cells [41]. The anti-asialo GM1 antibody (Wako USA, Richmond, Va.) was used to deplete mouse NK cells [42]. Gadolinium chloride (Sigma) was used to deplete mouse macrophages [43, 44]. Two well characterized saponin immunological adjuvants QS-21 and GPI-0100 were provided by Dr. P. Livingston (MSKCC).

### Mice

C57BL/6 and Balb/c mice (8 weeks old) were purchased from The Jackson Laboratory (Bar Harbor, Me.). Breeders of CS, CR3, FcγRIIb, FcγRIII knockout mice were obtained from The Jackson Laboratory. Fcer1g (FcRγ) knockout mice (deficient in the gamma chain subunit of the FcγRI, FcγRIII and FcεRI receptors) were obtained from Taconic (Hudson, N.Y.). CR2 knockout mice were kindly provided by Dr. M. Carroll (CBR, Harvard). Knockout mice were bred in the RARC of MSKCC. Mice were maintained in a pathogen-free vivarium according to NIH Animal Care guidelines. Experiments were done under the governance of an institutional protocol approved by the Memorial Sloan-Kettering Cancer Center (MSKCC) Institutional Animal Care and Use Committee. CD4 T cells were depleted by 200 μg L3T4 mAb iv on day-3, -2 and -1 before the start of the experiment and then once weekly throughout the experiment. Macrophages were depleted by GdCl₃ 0.5 mg ip on day-2 and -1 and once weekly thereafter. NK cells were depleted by 4 μl anti-asialo GM1 ip on day-6 and -3 and once weekly thereafter.

### Cell Lines

The EL4 cell line was established from lymphoma induced in a C57BL/6 mouse by 9,10-dimethyl-1,2-benzanthracene. It has been shown to express CD2 ganglioside [45]. The RVE tumor is a GD2-expressing leukemia cell line syngeneic for Balb/c mice (BALERVE provided to us by Dr. Elizabeth Stockert, MSKCC). EL4 and RVE cells were maintained in 10% FCS-RPMI. For vaccination, EL4 cells were washed three times in PBS, and 5×10⁴ cells were injected iv into the tail vein or 5×10⁵ were injected sc in the flank region for sc route. 2×10⁶ RVE cells were used for sc vaccination. EL4 cells were irradiated at 50 Gy in a ¹³⁷Cs γ-irradiator (Shepherd, San Fernando, Calif.) to obtain irradiated cells. For tumor cell challenge, EL4 cells were washed three times in PBS, and 5×10⁴ cells were injected iv into the tail vein.

### ELISA

ELISA was performed as described previously [46]. 96-well flat bottomed polyvinyl microtiter plates were coated with EL4 cells (50,000 cells/well), and dried at room temperature overnight; 0.01% gelatin in PBS was used as filler protein to saturate unbound sites. Mice serum diluted in PBS containing 0.03% BSA was allowed to react with the antigen plates at 37° C. for 2 h. A standard curve was constructed using serial dilutions of 3F8 mAb. After washing with PBS, the wells were reacted with peroxidase-conjugated affinity purified goat-anti-mouse IgG/IgM antibody (Southern Biotech, Birmingham, Ala.) diluted to 1:1000 in PBS containing 0.5% BSA at 4° C. for 1 h. After washing, the standard color reaction was performed. The absorbance was measured by an ELISA plate reader (MRX; Dynex, Chantilly, Va.). Based on the fitted regression curve of 3F8, the antibody titer of samples in μg/ml were obtained.

### Flow Cytometry

EL4 cells (5×10⁵) were incubated with 100 μl of 1:40 diluted mouse sera for 30 min on ice. After washing with 1% FBS in PBS, the cells were incubated with 100 μl of 1:50 diluted FITC-labeled goat antimouse IgG/IgM (Biosource, Camarillo, Calif.) for another 30 min on ice. The mean fluorescence intensity of the stained cells was quantitated by flow cytometry (EPICS Profile II; Coulter, Hialeah, Fla.). Antibody titers were calculated using the standard curve generated by serial dilutions of 3F8 mAb.

### Statistical Analyses

For serum antibody titers, statistical differences between groups were determined by analyzing means of replicates by two-tailed Student's t test. Differences in tumor-free survival were evaluated by log-rank analysis of Kaplan-Meier survival curves (GraphPad Prism 5.0).

### Results

### 3F8 mAb Treatment of Metastatic Tumor Induced Protective Immunity

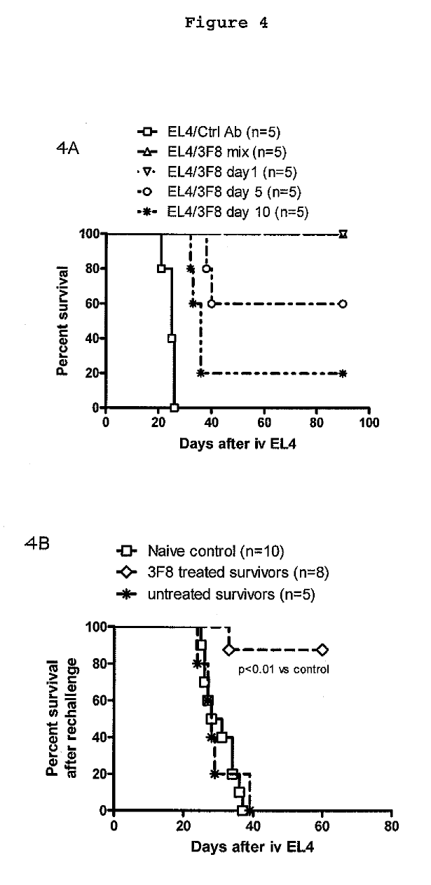

3F8 mAb is effective against EL4 metastatic tumors. Groups of mice (n=5 per group) received a single iv injection of 200 μg of 3F8 either mixed with or 1, 5, 10 days after EL4 tumor cells iv challenge. 100% of mice receiving 3F8 one day after challenge and 60% receiving 3F8 five days after challenge remained tumor free (FIG. 4, 1A). All mice treated with iv control HB11 antibody died by day 26. When surviving mice were re-challenged with iv EL4 tumor, 88% survived compared to 0% by day 39 in untreated control mice (p<0.01, FIG. 4, 1B) suggesting an effective anti-tumor memory response after successful 3F8 treatment.

Antibody Response to Whole Tumor EL4 Vaccine Mixed with 3F8 mAb

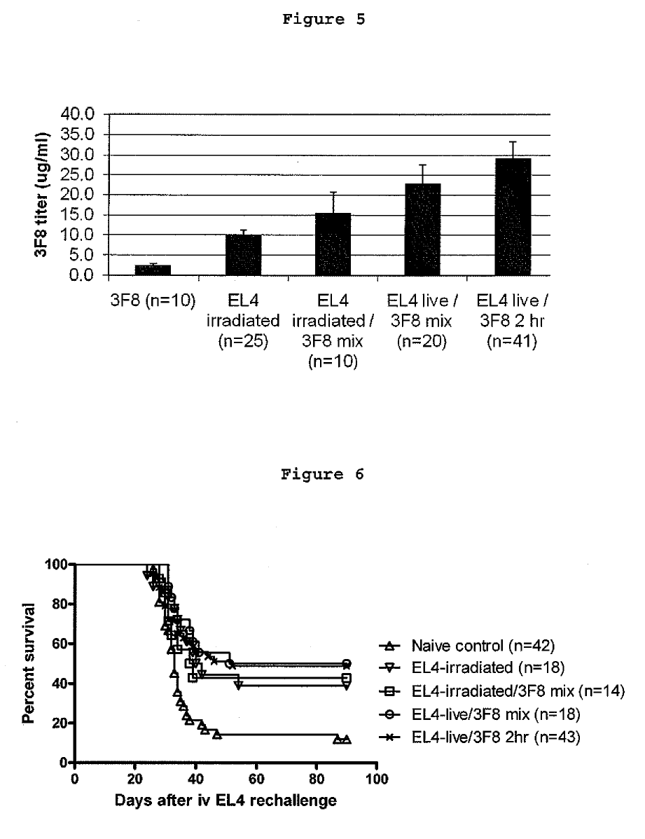

A combination of EL4 tumor cells and 3F8 mAb given intravenously was evaluated as a vaccine against EL4 tumor. C57B/6 mice were immunized intravenously through tail vein with 5×10⁴ live EL4 lymphoma tumor cells in the presence of 200 μg tumor-reactive 3F8 mAb. 3F8 was either directly mixed with tumor cells or given 2 hour after tumor cells to mimic a treatment setting. Irradiated tumor cells were included as a comparison. Mouse serum anti-EL4 tumor antibody titers were assayed by ELISA on EL4 cell plates. Live cells mixed with 3F8 or live cells treated with 3F8 in 2 hours all generated a significant serum anti-tumor antibody response compared with control mice receiving 3F8 only (p<0.01) and a trend of higher serum antibody response was obtained with live cells than irradiated cells (FIG. 5). Mice receiving live cells together with 3F8 (either direct mixture or 2 hours after tumor cell injection) survived significantly longer than control mice upon tumor iv re-challenge (p<0.05), comparable to irradiated cell or irradiated cells plus 3F8 (FIG. 6, Table 1).

TABLE 1
Summary of mice survival data after iv EL4 challenge
following immunization intravenously with EL4 tumor cells and
3F8 Ab

| | Death ratio | % Survival |
|---|---|---|
| Immunization | (<3 mos) | (>3 mos) |
| Naïve control | 37/42 | 11.9% |
| 3F8 iv | 7/9 | 22.2% |
| EL4-irradiated iv | 11/18 | 38.9% |
| EL4-irradiated + 3F8 | 8/14 | 42.9% |
| mix iv | | |
| EL4-irradiated + 3F8 | 5/5 | 0% |
| (2 hr) iv | | |
| EL4-live + 3F8 mix iv | 9/18 | 50.0% |
| EL4-live + 3F8 (2 hr) iv | 22/43 | 48.8% |

*Death ratio = number of mice dead/total number of mice treated

Subcutaneous Whole Tumor Vaccine Mixed with 3F8 mAb and Yeast β-Glucan

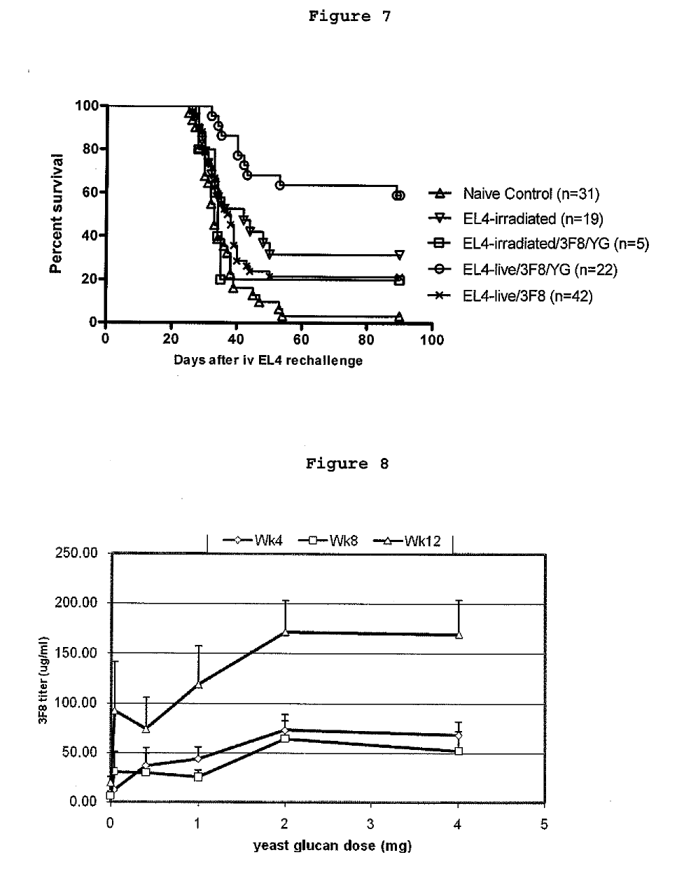

C57B/6 mice were immunized sc with live EL4 lymphoma tumor cells (5×10⁵) in the presence of tumor-reactive 3F8 (50 μg) plus yeast β-glucan (0.1-4 mg). Mouse serum anti-EL4 antibody titers were assayed by ELISA. Similarly to the iv vaccine route, live cells mixed with 3F8 generated a significantly higher anti-tumor antibody response compared with control mice receiving 3F8 Ab only (p<0.01) and again a trend towards higher Ab response was obtained with live cells than irradiated cells (data not shown). Mice receiving live cells and 3F8 survived significantly longer than control mice upon re-challenge (p<0.05, FIG. 4). More importantly, when yeast β-glucan is included as an adjuvant in the immunization, substantial Ab response and tumor protection were achieved. Mice receiving live cells mixed with 3F8 and yeast β-glucan survived significantly longer than mice receiving live cells and 3F8 upon re-challenge (p<0.001, FIG. 7). The dose of yeast β-glucan was found to correlate with antibody titer against EL4 tumor cells (FIG. 8) and tumor protection (Table 2) upon subsequent re-challenge.

TABLE 2
Summary of mice survival data after iv EL4 challenge
following immunization subcutaneously with EL4 tumor cells,
3F8 Ab and yeast β-glucan

| | Death ratio | % Survival |
|---|---|---|
| Prior treatment/immunization | (<3 mos) | (>3 mos) |
| Naïve control | 30/31 | 3.2% |
| EL4-irradiated sc | 13/19 | 31.6% |
| EL4-irradiated + 3F8 + yeast | 4/5 | 20.0% |
| glucan mix sc | | |
| EL4-live + 3F8 + yeast glucan 4 mg | 2/5 | 60.0% |
| mix sc | | |
| EL4-live + 3F8 + yeast glucan 2 mg | 9/22 | 59.1% |
| mix sc | | |
| EL4-live + 3F8 + yeast glucan 1 mg | 3/5 | 40.0% |
| mix sc | | |
| EL4-live + 3F8 + yeast glucan 0.4 mg | 17/23 | 26.1% |
| mix sc | | |
| EL4-live + 3F8 + yeast glucan <0.4 mg | 17/20 | 15.0% |
| mix sc | | |
| EL4-live + 3F8 + mix sc | 33/42 | 21.4% |

*Death radio = number of mice dead/total number of mice treated

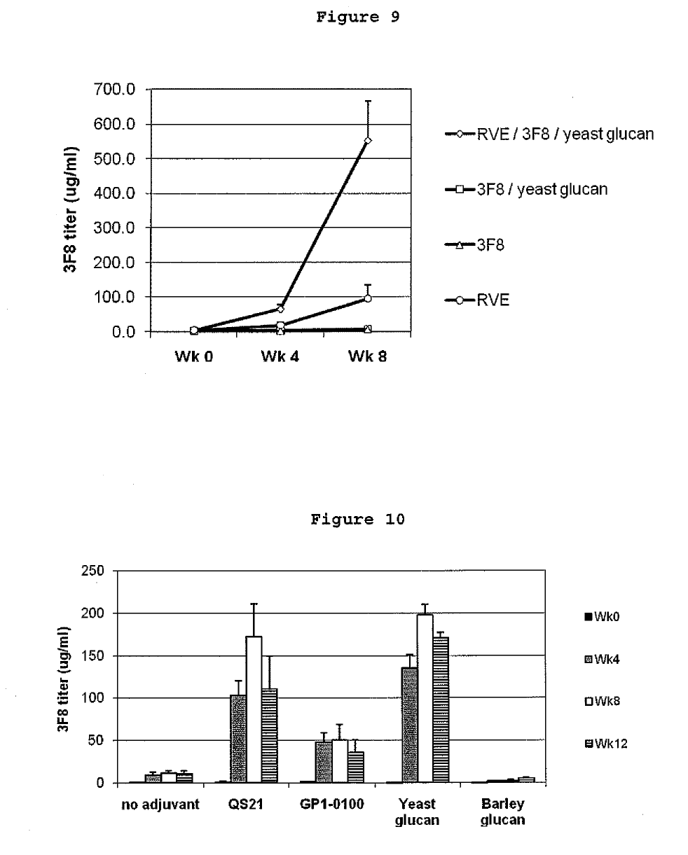

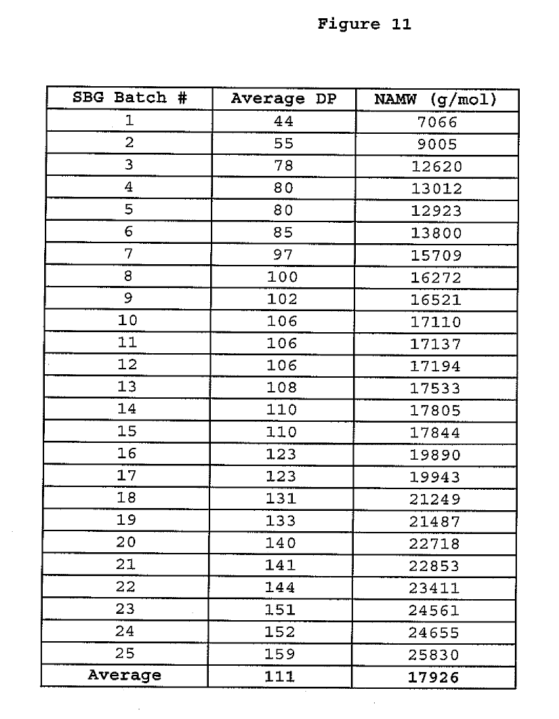

Anti-EL4 tumor response induced by sc EL4/3F8/yeast β-glucan immunization is not against GD2 because mice serum did not react with the GD2-positive neuroblastoma cell line LAN-1. When another GD2-positive lymphoma RVE cell was mixed with 3F8 and yeast β-glucan as a sc vaccine in the Balb/c mice, a strong anti-tumor antibody response was again induced (FIG. 9).

Comparing the Yeast β-Glucan with Other Adjuvants

The effects of several different adjuvants were compared in the sc EL4/3F8 vaccine regimen. QS21 and GPI-0100 are two saponin immunological adjuvants known to have maximal tolerated doses at 20 μg and 200 μg, respectively [47]. Yeast glucan has an adjuvant effect comparable to QS21 but better than GPI-0100 (FIG. 10).

Receptor Dependence for this Whole Tumor/Antibody/β-Glucan Vaccine Efficacy

The importance of CD4 T cells, macrophages and NK cells after their depletion in the induction of antibody response and in tumor protection was tested. The efficacy of the whole cell tumor vaccine regimen in wild type mice was compared with that in knock-out mice. These mice were genetically deficient in either one of the following: C3, CR2, CR3, FcRγ, FcγRIIB, or FcγRIII. The 3F8 and yeast glucan adjuvant effect required CD4 T cell, macrophage and CR2 but did not require C3, CR3 or FcγRs (Table 3).

TABLE 3
Summary of anti-tumor antibody response and tumor protection
in CD4 T cell, macrophage, and NK cell-depleted mice and C3,
CR2, CR3, FcRγ, FcγRIIB and FcγRIII-deficient mice.

| | Anti-EL4 | Protection from iv |
|---|---|---|
| | antibody response | EL4 challenge |
| Wild-type | | |
| Live vs irradiated | Yes | Yes |
| EL 4 cells | | |
| 3F8 + EL4 cells vs | Yes | Yes |
| 3F8 | | |
| Cell depleted | | |
| CD4− | No | No |
| Macrophage− | No | No |
| NK− | Yes | No |
| Knockout mice | | |
| C3−/− | Yes | Yes |
| CR2−/− | No | NA (susceptible to |
| | | EL4) |
| CR3−/− | Yes | Yes |
| FcRγ−/− | Yes | No |
| FcγRIIB−/− | Yes | NA (resistant to |
| | | EL4) |
| FcγRIII−/− | Yes | Yes |

### Discussion

This study demonstrates that whole cell tumor vaccine in combination with IgG3 mAb induced an anti-tumor antibody response which is protective against tumor re-challenge. This effect is further enhanced by yeast β-glucan.

Irradiated (dead) tumor cells can be used as a vaccine by inducing anti-tumor antibody response. The results indicated that 3F8 antibody together with live EL4 tumor cell (either mixed with or given 2 hours later) induce an antibody response that is protective against EL4 tumor re-challenge that is comparable to irradiated EL4 tumor cells. 3F8 and yeast β-glucan mixed with live EL4 tumor cells generate significantly better antibody response and better survival than irradiated tumor cells.

Nascent endogenous anti-tumor antibodies in the naïve mouse are clearly inadequate because they cannot protect mice from tumor challenge. Dead tumor cells could induce antibody response, but this response was much enhanced when 3F8 was administered and when live cells were present, suggesting that mAb treatment when there is active tumor may play an active role in inducing tumor immunity. It is likely that induced antibodies will bind epitopes distinct from GD2 (the target antigen for 3F8), providing additive effects in promoting antibody-dependent tumor cell cytotoxicity or the afferent arms of T-cell dependent tumor immunity.

Previous report shows that barley glucan, being basically a linear β-1,3-1,4-glucan, has no effect on human DCs. In contrast, Ganoderma lucidum (GL, Lingzhi) polysaccharides are more immunogenic [36].

The data provided here show immune sensitization during treatment with antitumor antibodies. The induction of endogenous humoral immunity suggests that therapeutic antibodies not only provide passive immunotherapy through antibody-dependent tumor cell cytotoxicity but also can promote active immunity.

### EXAMPLE 3

### Phase I Study of Orally Administered Yeast β-Glucan

In this phase I study, patients with refractory or recurrent metastatic stage 4 neuroblastoma were recruited. They all received anti-GD2 antibody 3F8 at 10 mg/m²/day for a total of 10 days, while being given oral yeast β-glucan. The dose of yeast β-glucan was escalated in cohorts of 3-6 patients (10, 20, 40, 80, 100, 120 mg/kg/dose). Eighteen patients have been registered. There was no dose limiting toxicities (DLTs).

Three (3) patients were registered and treated at 10 mg/kg dose level. These patients have completed all four cycles of treatment. Two of these three patients showed a minor response. One patient had progressive disease.

Three (3) patients were registered and treated at the 20 mg/kg dose level. One (1) completed all four cycles with an objective response and had an additional four cycles of treatment approved by IRB. He is now completing cycle 6. One (1) patient has completed all four cycles and undergoing extent of disease evaluation. One (1) patient completed three (3) cycles of treatment and then developed human anti-mouse response (HAMA). Extent of disease evaluation is pending.

Three (3) patients were registered and treated at the 40 mg/kg dose level. One (1) completed all four cycles of treatment. Extent of disease evaluation at the end of four cycles revealed progression of disease. One (1) patient completed one cycle of treatment. Extent of disease evaluation after one cycle revealed progressive disease. One (1) patient is now completing cycle 3.

Six (6) patients have been registered and treated at the 80 mg/kg dose level. One (1) of the patients completed all four cycles of treatment and extent of disease evaluation and had a very good partial response (VGPR). One (1) patient completed two cycles. Extent of disease evaluation after two cycles revealed progressive disease. Two (2) patients completed only one cycle of treatment and had progressive disease after one cycle. One (1) patient is receiving cycle 2 of treatment. One (1) patient has completed one cycle of treatment. The latter two patients continue on protocol.

Three (3) patients were registered and treated at the 100 mg/kg dose level. There were no dose limiting toxicities. One patient (1) has progressed. One (1) patient achieved a complete remission of marrow disease. The last patient was still too early to be evaluated for response. The latter two patients continue on protocol.

### REFERENCES

1. Diller, I. C. et al., 1963, “The effect of yeast polysaccharides on mouse tumors”, Cancer Res, 23: 201-208.
2. Sveinbjornsson, B. et al., 1998, “Inhibition of establishment and growth of mouse liver metastases after treatment with interferon gamma and beta-1,3-D-Glucan”, Hepatology, 27(5): 1241-1248.
3. Niimoto, M. et al., 1988, “Postoperative adjuvant immunochemotherapy with mitomycin C, futraful, and PSK for gastric cancer. An analysis of data on 579 patients followed for five years”, Japanese Journal of Surgery, 18: 681-686.
4. Nakazato, H. et al., 1994, “Efficacy of immunochemotherapy as adjuvant treatment after curative resection of gastric cancer. Study Group of Immunochemotherapy with PSK for Gastric Cancer”, Lancet, 343: 1122-1126.
5. Torisu, M. et al., 1990, “Significant prolongation of disease-free period gained by oral polysacharide K (PSK) administration after curative surgical operation of colorectal cancer”, Cancer Immunol. Immunother., 31: 261-268.
6. Mitomi, T. et al., 1992, “Randomized, controlled study on adjuvant immunochemotherapy with PSK in curatively resected colorectal cancer. The cooperative study group of surgical adjuvant immunochemotherapy for cancer of colon and rectum (Kanagawa)”, Dis. Colon Rectum, 35: 123-130.
7. Ogoshi, K. et al., 1995, “Immunotherapy for esophageal cancer. A randomized trial in combination with radiotherapy and radiochemotherapy. Cooperative study group for esophageal cancer in Japan”, American Journal of Clinical Oncology, 18: 216-222.
8. Toi, M. et al., 1992, “Randomized adjuvant trial to evaluate the addition of tamoxifen and PSK to chemotherapy in patients with primary breast cancer. 5-year results from the Nishi-Nippon group of the adjuvant chemoendocrine therapy for breast cancer organization”, Cancer, 70: 2475-2483.
9. Lino, Y. et al., 1995, “Immunochemotherapies versus chemotherapy as adjuvant treatment after curative resection of operable breast cancer”, Anticancer Res., 15: 2907-2911.
10. Ohno, R., et al., 1984, “A randomized trial of chemoimmunotherapy of acute nonlymphocytic leukemia in adults using a protein-bound polysaccharide preparation”, Cancer Immunol. Immunother., 18: 149-154.
11. Fujimoto, S. et al., 1991, “Clinical outcome of postoperative adjuvant immunochemotherapy with sizofuran for patients with resectable gastric cancer: a randomised controlled study”, Eur. J. Cancer, 27: 1114-1118.
12. Furue, H. et al., 1985, “Clinical evaluation of schizophyllan (SPG) in advanced gastric cancer (the second report)—a randomized controlled study”, Gan To Kagaku Ryoho, 12: 1272-1277.
13. Nakao, I., et al., 1983, “Clinical evaluation of schizophyllan (SPG) in advanced gastric cancer—a randomized comparative study by an envelop method”, Jpn. J. Cancer Chemother., 10: 1146-1159.
14. Okamura, K. et al., 1989, “Clinical evaluation of sizofuran combined with irradiation in patients with cervical cancer. A randomized controlled study; a five-year survival rate”, Biotherapy, 1: 103-107.
15. Mayell, M., 2001, “Maitake extracts and their therapeutic potential”, Altern. Med. Rev., 6: 48-60.
16. Engstad, R. and J. Raa., 1999, “Immune-stimulation improving health and performance”, Feed Magazine (Kraftfutter), 7-8: 261-266.
17. Nicolosi, R. et al., 1999, “Plasma Lipid changes after supplementation with beta-glucan fiber from yeast”, Am. J. Clin. Nutr. 70: 208-212.
18. Kernodle, D. D. et al., 1998, “Prophylactic Anti-Infective Activity of Poly-[1-6]-D-Glucopyranosyl-[1-3]-D-Glucopyranose Glucan in a Guinea Pig Model of Staphylococcal Wound Infection”, Antimicrobial Agents and Chemotherapy, 42: 545-549.
19. Seljelid, R. 1986. “A water soluble aminated β-1,3-D-glucose derivative caused regression of solid tumors in mice. Bioscience Reports 6:845-852
20. Williams, D. L. et al., 1991, “Development, physicochemical characterization and preclinical efficacy evaluation of a water soluble glucan sulfate derived from Saccharomyces cerevisiae”, Immunopharmacology, 22: 139-155.
21. Iannello, A. et al., 2005, “Role of antibody-dependent cell-mediated cytotoxicity in the efficacy of therapeutic anti-cancer monoclonal antibodies”, Cancer Metastasis Reviews, 24(4): 487-499.
22. Trauth, B. C., et al., 1989, “Monoclonal antibody-mediated tumor regression by induction of apoptosis”, Science, 245: 301-305.
23. Dillman, R. O., 2001, “Monoclonal antibodies in the treatment of malignancy: basic concepts and recent developments”, Cancer Investigation, 19(8): 833-841.
24. Clynes, R. A. et al., 2000, “Inhibitory Fc receptors modulate in vivo cytoxicity against tumor targets”, Nature Medicine, 6(4): 443-446.
25. Cartron, G. et al., 2002, “Therapeutic activity of humanized anti-CD20 monoclonal antibody and polymorphism in IgG Fc receptor FcgammaRIIIa gene”, Blood, 99(3): 754-758.
26. Weng, W. K. et al., 2003, “Two immunoglobulin G fragment C receptor polymorphisms independently predict response to rituximab in patients with follicular lymphoma”, J. Clinical Oncology, 21(21): 3940-3947.
27. Cheung, N. K., et al., 2006, “FCGR2A polymorphism is correlated with clinical outcome after immunotherapy of neuroblastoma with anti-GD2 antibody and granulocyte macrophage colony-stimulating factor”, J. Clinical Oncology, 24(18): 2885-2890.
28. Rafiq, K. et al., 2002, “Immune complex-mediated antigen presentation induces tumor immunity”, J. Clinical Investigation, 110(1): 71-79.
29. Dhodapkar, K. M., et al., 2002, “Antitumor monoclonal antibodies enhance cross-presentation of cellular antigens and the generation of myeloma-specific killer T cells by dendritic cells”, J. Experimental Medicine, 195(1): 125-133.
30. Kushner, B. H. et al., 2001, “Phase IT trial of the anti-G(D2) monoclonal antibody 3F8 and granulocyte-macrophage colony-stimulating factor for neuroblastoma”, J. Clin. Oncol., 19(22): 4189-94.
31. Cheung, N. K. et al., 1998, “Anti-G(D2) antibody treatment of minimal residual stage 4 neuroblastoma diagnosed at more than 1 year of age”, J. Clin. Oncol., 16(9): 3053-60.
32. Diaz de Stahl, T. et al., 2003, “A role for complement in feedback enhancement of antibody responses by IgG3”, J. Experimental Medicine, 197(9): 1183-1190.
33. Zhang, H. et al., 1998, “Antibodies against GD2 ganglioside can eradicate syngeneic cancer micrometastases”, Cancer Research, 58(13): 2844-2849.
34. Imai, M. et al., 2005, “Complement-mediated mechanisms in anti-GD2 monoclonal antibody therapy of murine metastatic cancer”, Cancer Research, 65(22): 10562-10568.
35. Yoshitomi, H. et al., 2005, “A role for fungal {beta}-glucans and their receptor Dectin-1 in the induction of autoimmune arthritis in genetically susceptible mice”, J. Experimental Medicine, 201(6): 949-960.
36. Chan, W. K. et al., 2007, “Response of human dendritic cells to different immunomodulatory polysaccharides derived from mushroom and barley”, International Immunology, 19(7): 891-899.
37. Cheung, N. K. et al., 2002, “Oral (1-->3), (1-->4)-beta-D-glucan synergizes with antiganglioside GD2 monoclonal antibody 3F8 in the therapy of neuroblastoma”, Clinical Cancer Research, 8(5): 1217-1223.
38. Cheung, N. K. et al., 2002, “Orally administered beta-glucans enhance anti-tumor effects of monoclonal antibodies”, Cancer Immunology Immunotherapy, 51(10): 557-564.
39. Hong, F. et al., 2003, “Beta-glucan functions as an adjuvant for monoclonal antibody immunotherapy by recruiting tumoricidal granulocytes as killer cells”, Cancer research, 63(24): 9023-9031.
40. Cheung, N. K. et al., 1985, “Monoclonal antibodies to a glycolipid antigen on human neuroblastoma cells”, Cancer Research, 45(6): 2642-2649.
41. Mody, C. H. et al., 1990, “Depletion of CD4+ (L3T4+) lymphocytes in vivo impairs murine host defense to Cryptococcus neoformans”, J. Immunology 144(4): 1472-1477.
42. Yoshino, H. et al., 2000, “Natural killer cell depletion by anti-asialo GM1 antiserum treatment enhances human hematopoietic stem cell engraftment in NOD/Shi-scid mice”, Bone Marrow Transplantation, 26(11): 1211-1216.
43. Adding, L. C. et al., 2001, “Basic experimental studies and clinical aspects of gadolinium salts and chelates”, Cardiovascular Drug Reviews, 19(1): 41-56.
44. Roland, C. R. et al., 1999, “Gadolinium chloride inhibits lipopolysaccharide-induced mortality and in vivo prostaglandin E2 release By splenic macrophages”, J. of Gastrointestinal Surgery, 3(3): 301-307.
45. Zhao, X. J. and N. K. Cheung, 1995, “GD2 oligosaccharide: target for cytotoxic T lymphocytes”, Journal Experimental Medicine, 182(1): 67-74.
46. Cheung, N. K. et al., 1994, “Antibody response to murine anti-GD2 monoclonal antibodies: correlation with patient survival”, Cancer research, 54(8): 2228-2233.
47. Livingston, P. O. et al., 1994, “Phase 1 trial of immunological adjuvant QS-21 with a GM2 ganglioside-keyhole limpet haemocyanin conjugate vaccine in patients with malignant melanoma”, Vaccine, 12(14): 1275-1280.

What is claimed is:

1. A composition for enhancing protective immunity against cancer in a subject, comprising:
(a) a vaccine comprising an antibody and one or more components selected from the group consisting of whole tumor cells, tumor cell lysates, tumor cell derived RNAs, tumor cell derived proteins, tumor cell derived peptides, tumor cell derived carbohydrate, tumor cell derived lipids, tumor cell derived DNA sequences, and gene modified tumor cells; and
(b) a β-glucan having β-(1,3) side chains.

2. The composition of claim 1, wherein the β-glucan is derived from yeast.

3. The composition of claim 1, wherein the side chains of said β-glucan are attached to a β-(1,3) backbone via β-(1,6) linkages.

4. The composition of claim 1, wherein the β-glucan has a numerical average molecular weight from about 6 kDa to about 30 kDa, and a weighted average molecular weight of 2×10⁵-3×10⁶ g/mol, and wherein one or more β-glucan molecules form a higher order conformation, resulting in gelling and high viscosity profile.

5. The composition of claim 1, wherein said β-glucan is capable of priming or inducing secretion of cytokines, chemokines or growth factors.

6. The composition of claim 1, wherein the antibody binds to the Fc receptor or activates complement.

7. The composition of claim 1, wherein the antibody is selected from the group consisting of anti-CEA antibody, anti-CD20 antibody, anti-tenascin antibody, anti-TAG-72 antibody, M195 antibody, DACLUZIMAB, R24 antibody, HERCEPTIN, RITUXIMAB, 528 antibody, IgG antibody, IgM antibody, IgA antibody, C225 antibody, EPRATUZUMAB, 3F8 antibody, an antibody directed at the epidermal growth factor receptor, anti-ganglioside antibody, anti-GD3 antibody, and anti-GD2 antibody.

8. The composition of claim 1, wherein the antibody binds to cancer cells expressing an antigen selected from the group consisting of CD20, HER2, EGFR, GD2, and GD3.

9. A method of enhancing protective immunity against cancer in a subject, comprising the steps of:
(a) administering to the subject a vaccine comprising an antibody, and
(b) administering to the subject a β-glucan having β-(1,3) side chains;
wherein cancer growth in said subject is treated or prevented.

10. The method of claim 9, wherein the antibody is an opsonising antibody.

11. The method of claim 9, wherein the vaccine further comprises one or more components selected from the group consisting of whole tumor cells, tumor cell lysates, tumor cell derived RNAs, tumor cell derived proteins, tumor cell derived peptides, tumor cell derived carbohydrate, tumor cell derived lipids, tumor cell derived DNA sequences, and gene modified tumor cells.

12. The method of claim 9, wherein the β-glucan is derived from yeast.

13. The method of claim 9, wherein the side chains of said β-glucan are attached to a β-(1,3) backbone via β-(1,6) linkages.

14. The method of claim 9, wherein said β-glucan has a numerical average molecular weight from about 6 kDa to about 30 kDa, and a weighted average molecular weight of 2×10⁵-3×10⁶ g/mol, and wherein one or more β-glucan molecules form a higher order conformation, resulting in gelling and high viscosity profile.

15. The method of claim 9, wherein said β-glucan is capable of priming or inducing secretion of cytokines, chemokines or growth factors.

16. The method of claim 9, wherein the cancer is neuroblastoma, melanoma, non-Hodgkin's lymphoma, Epstein-Barr related lymphoma, Hodgkin's lymphoma, retinoblastoma, small cell lung cancer, brain tumors, leukemia, epidermoid carcinoma, prostate cancer, renal cell carcinoma, transitional cell carcinoma, breast cancer, ovarian cancer, lung cancer colon cancer, liver cancer, stomach cancer, and other gastrointestinal cancers.

17. The method of claim 9, wherein the antibody binds to the Fc receptor or activates complement.

18. The method of claim 9, wherein the antibody is selected from the group consisting of anti-CEA antibody, anti-CD20 antibody, anti-tenascin antibody, anti-TAG-72 antibody, M195 antibody, DACLUZIMAB, R24 antibody, HERCEPTIN, RITUXIMAB, 528 antibody, IgG antibody, IgM antibody, IgA antibody, C225 antibody, EPRATUZUMAB, 3F8 antibody, an antibody directed at the epidermal growth factor receptor, anti-ganglioside antibody, anti-GD3 antibody, and anti-GD2 antibody.

19. The method of claim 9, wherein the antibody binds to cancer cells expressing an antigen selected from the group consisting of CD20, HER2, EGFR, GD2, and GD3.

20. The method of claim 9, wherein the vaccine and glucan are administered orally, intravenously, subcutaneously, intramuscularly, intraperitoneally, intranasally or transdermally, concurrently or sequentially.
